(19) United States
(12) Patent Application Publication
(10) Pub. No.: US 2007/0232681 A1
(43) Pub. Date: Oct. 4, 2007

(54) Compounds Having Crth2 Antagonist Activity

(75) Inventors: David Middlemiss, Bishop's Stortford (GB); Mark Richard Ashton, Abingdon (GB); Edward Andrew Boyd, Abingdon (GB); Frederick Arthur Brookfield, Abingdon (GB); Richard Edward Armer, Abingdon (GB); Eric Roy Pettipher, Abingdon (GB)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: Oxagen Limited, Abingdon (GB)

(21) Appl. No.: 10/573,670

(22) PCT Filed: Oct. 13, 2004
(86) PCT No.: PCT/GB04/04336

§ 371 Date: Jan. 8, 2007

(30) Foreign Application Priority Data
Oct. 14, 2003 (GB) 0324084.3
Jan. 14, 2004 (GB) 0400716.7

(51) Int. Cl. A61K 31/40; A61K 31/405; C07D 209/04; C07D 209/34

(52) U.S. Cl. 514/415; 514/418; 548/469; 548/486

(57) ABSTRACT

Compounds of general formula (I): 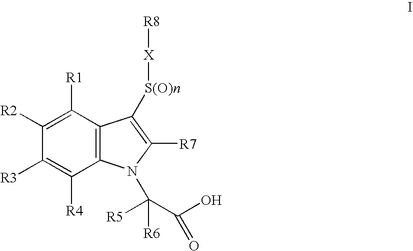 wherein
R¹, R², R³ and R⁴ are independently hydrogen, halo, C₁-C₆ alkyl, —O(C₁-C₆ alkyl), —CON(R⁹)₂, —SOR⁹, —SO₂R⁹, —SO₂N(R⁹)₂, —N(R⁹)₂, —NR⁹COR⁹, —CO₂R⁹, —COR⁹, —SR⁹, —OH, —NO₂ or —CN;
   each R⁹ is independently hydrogen or C₁-C₆ alkyl;
R⁵ and R⁶ are each independently hydrogen, or C₁-C₆ alkyl or together with the carbon atom to which they are attached form a C₃-C₇ cycloalkyl group;
R⁷ is hydrogen or C₁-C₆ alkyl n is 1 or 2;
X is a bond or, when n is 2, X may also be a NR⁹ group;
   wherein R⁹ is as defined above;
when X is a bond R⁸ is C₁-C₆ alkyl, C₂-C₆ alkenyl, C₂-C₆ alkynyl, biphenyl or a 9-14 membered bicyclic or tricyclic heteroaryl group;
when X is a NR⁹ group R⁸ may additionally be phenyl, naphthyl or a 5-7 membered heteroaromatic ring; and
the R⁸ group is optionally substituted with one or more substituents selected from halo, C₁-C₆ alkyl, —O(C₁-C₆)alkyl, aryl, —O-aryl, heteroaryl, —O-heteroaryl, —CON(R⁹)₂, —SOR⁹, —SO₂R⁹, SO₂N(⁹)₂, —N(⁹)₂, —NR⁹COR⁹, —CO₂R⁹, —COR⁹, —SR⁹, —OH, —NO₂ or —CN;
   wherein R⁹ is as defined above; and their pharmaceutically acceptable salts, hydrates, solvates, complexes and prodrugs are useful in the treatment of allergic diseases such as asthma, allergic rhinitis and atopic dermatitis.

The present invention relates to compounds which are useful as pharmaceuticals, to methods for preparing these compounds, compositions containing them and their use in the treatment and prevention of allergic diseases such as asthma, allergic rhinitis and atopic dermatitis and other inflammatory diseases mediated by prostaglandin D₂ (PGD₂) acting at the CRTH2 receptor on cells including eosinophils, basophils and Th2 lymphocytes.

PGD₂ is an eicosanoid, a class of chemical mediator synthesised by cells in response to local tissue damage, normal stimuli or hormonal stimuli or via cellular activation pathways. Eicosanoids bind to specific cell surface receptors on a wide variety of tissues throughout the body and mediate various effects in these tissues. PGD₂ is known to be produced by mast cells, macrophages and Th2 lymphocytes and has been detected in high concentrations in the airways of asthmatic patients challenged with antigen (Murray et al, (1986), N. Engl. J. Med. 315: 800-804). Instillation of PGD₂ into airways can provoke many features of the asthmatic response including bronchoconstriction (Hardy et al, (1984) N. Engl. J. Med. 311: 209-213; Sampson et al, (1997) Thorax 52: 513-518) and eosinophil accumulation (Emery et al, (1989) J. Appl. Physiol. 67: 959-962).

The potential of exogenously applied PGD₂ to induce inflammatory responses has been confirmed by the use of transgenic mice overexpressing human PGD₂ synthase which exhibit exaggerated eosinophilic lung inflammation and Th2 cytokine production in response to antigen (Fujitani et al, (2002) J. Immunol. 168: 443449).

The first receptor specific for PGD₂ to be discovered was the DP receptor which is linked to elevation of the intracellular levels of cAMP. However, PGD₂ is thought to mediate much of its proinflammatory activity through interaction with a G protein-coupled receptor termed CRTH2 (chemoattractant receptor-homologous molecule expressed on Th2 cells) which is expressed by Th2 lymphocytes, eosinophils and basophils (Hirai et al, (2001) J. Exp. Med. 193: 255-261, and EP0851030 and EP-A-1211513 and Bauer et al, EP-A-1170594). It seems clear that the effect of PGD₂ on the activation of Th2 lymphocytes and eosinophils is mediated through CRTH2 since the selective CRTH2 agonists 13,14 dihydro-15-keto-PGD₂ (DK-PGD₂) and 15R-methyl-PGD₂ can elicit this response and the effects of PGD₂ are blocked by an anti-CRTH2 antibody (Hirai et al, 2001; Monneret et al, (2003) J. Pharmacol. Exp. Ther. 304: 349-355). In contrast, the selective DP agonist BW245C does not promote migration of Th2 lymphocytes or eosinophils (Hirai et al, 2001; Gervais et al, (2001) J. Allergy Clin. Immunol. 108: 982-988). Based on this evidence, antagonising PGD₂ at the CRTH2 receptor is an attractive approach to treat the inflammatory component of Th2-dependent allergic diseases such as asthma, allergic rhinitis and atopic dermatitis.

EP-A-1170594 suggests that the method to which it relates can be used to identify compounds which are of use in the treatment of allergic asthma, atopic dermatitis, allergic rhinitis, autoimmune disease, reperfusion injury and a number of inflammatory conditions, all of which are mediated by the action of PGD₂ at the CRTH2 receptor.

Compounds which bind to CRTH2 are taught in WO-A-03066046 and WO-A-03066047. These compounds are not new but were first disclosed, along with similar compounds, in GB 1356834, GB 1407658 and GB 1460348, where they were said to have anti-inflammatory, analgesic and antipyretic activity. WO-A-03066046 and WO-A-03066047 teach that the compounds to which they relate are modulators of CRTH2 receptor activity and are therefore of use in the treatment or prevention of obstructive airway diseases such as asthma, chronic obstructive pulmonary disease (COPD) and a number of other diseases including various conditions of bones and joints, skin and eyes, GI tract, central and peripheral nervous system and other tissues as well as allograft rejection. The compounds described in these documents are indoles with a carboxylic acid group is at the 3-position of the indole ring system a quinoline, quinazoline or benzothiazole group at the 1-position.

The present invention relates to novel compounds which bind to CRTH2 and which will therefore also be useful in the treatment of diseases and conditions mediated by the activity of PGD₂ at the CRTH2 receptor.

In the present invention there is provided a compound of general formula (I) 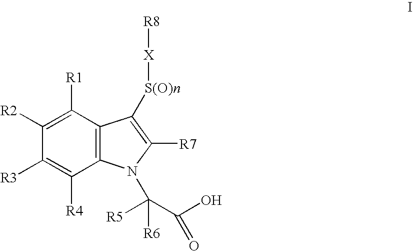
wherein
R¹, R², R³ and R⁴ are independently hydrogen, halo, C₁-C₆ alkyl, —O(C₁-C₆ alkyl), —CON(R⁹)₂, —SOR⁹, —SO₂R⁹, —SO₂N(R⁹)₂, —N(R⁹)₂, —NR⁹COR⁹, —CO₂R⁹, —COR⁹, —SR⁹, —OH, —NO₂ or —CN;
   each R⁹ is independently hydrogen or C₁-C₆ alkyl;
R⁵ and R⁶ are each independently hydrogen, or C₁-C₆ alkyl or together with the carbon atom to which they are attached form a C₃-C₇ cycloalkyl group;
R⁷ is hydrogen or C₁-C₆ alkyl
n is 1 or 2;
X is a bond or, when n is 2, X may also be a NR⁹ group;
   wherein R⁹ is as defined above;
when X is a bond R⁸ is C₁-C₆ alkyl, C₂-C₆ alkenyl, C₂-C₆ alkynyl, biphenyl or a 9-14 membered bicyclic or tricyclic heteroaryl group;
when X is a NR⁹ group R⁸ may additionally be phenyl, naphthyl or a 5-7 membered heteroaromatic ring; and
the R⁸ group is optionally substituted with one or more substituents selected from halo, C₁-C₆ alkyl, —O(C₁-C₆)alkyl, aryl, —O-aryl, heteroaryl, —O-heteroaryl, —CON(R⁹)₂, —SOR⁹, —SO₂R⁹, SO₂N(R⁹)₂, —N(R⁹)₂, —NR⁹COR⁹, —CO₂R⁹, —COR⁹, —SR⁹, —OH, —NO₂ or —CN;
   wherein R⁹ is as defined above;
      or a pharmaceutically acceptable salt, hydrate, solvate, complex or prodrug thereof.

The compounds of general formula (I) are antagonists of PGD₂ at the CRTH2 receptor and will therefore be useful in the treatment of conditions which are mediated by PGD₂ binding to CRTH2. These include allergic diseases, asthmatic conditions and inflammatory diseases, examples of which are allergic asthma, perennial allergic rhinitis, seasonal allergic rhinitis, atopic dermatitis, contact hypersensitivity (including contact dermatitis), conjunctivitis, especially allergic conjunctivitis, eosinophilic bronchitis, food allergies, eosinophilic gastroenteritis, inflammatory bowel disease, ulcerative colitis and Crohn's disease, mastocytosis and also other PGD₂-mediated diseases, for example autoimmune diseases such as hyper IgE syndrome and systemic lupus erythematus, psoriasis, acne, multiple sclerosis, allograft rejection, reperfusion injury, chronic obstructive pulmonary disease, as well as rheumatoid arthritis, psoriatic arthritis and osteoarthritis.

Similar, but not identical, compounds are disclosed in WO-A-9950268. These compounds differ from those of the present invention in that they do not contain a sulfone/sulfonamide moiety attached to the 3-position of the indole ring. In addition, they are not taught to be useful in the treatment of conditions such as asthma and allergic conditions, which are mediated by PGD₂. Rather, they are said to be of use in the treatment of complications arising from diabetes mellitus.

PL 65781 and JP 43-24418 also relate to indole derivatives. However, the compounds disclosed in both of these documents differ from the compounds of the present application in that they are indole N-sulfonamides rather than 3-sulfones or 3-sulfonamides like the compounds of the present invention. The compounds disclosed in PL 65781 and JP 43-24418 are similar in structure to indomethacin and, like indomethacin, are said to have anti-inflammatory and antipyretic activity. Thus, although this may not have been appreciated at the time when these documents were published, the compounds they describe are COX inhibitors, an activity which is quite different from that of the compounds of the present invention. Indeed, COX inhibitors are contraindicated in the treatment of many of the diseases and conditions, for example asthma and inflammatory bowel disease, for which the compounds of the present invention are useful, although they may sometimes be used to treat arthritic conditions.

Compounds which bind to the CRTH2 receptor are disclosed in WO-A-03/097042 and WO-A-03/097598. These compounds are indole acetic acids but in WO-A-03/097042 the indole system is fused at the 2-3 positions to a 5-7 membered carbocyclic ring. In WO-A-03/097598 there is a pyrrolidine group at the indole 3-position.

WO-A-03/101981 and WO-A-03/101961 both relate to CRTH2 antagonists. The compounds described in WO-A-03/101961 are similar in structure to the compounds of the present invention in which X is a bond. They differ from the compounds of general formula (I) because there is an —S— group linked to the indole 3-position in place of the SO or SO₂ group of the compounds of general formula (I). In addition, the group equivalent to the R⁸ group in the compounds of general formula (I) is an aryl or heteroaryl group. There are no aliphatic substitutents at this position as with the compounds of general formula (I). It has surprisingly been found that although these compounds have high intrinsic activity, they are less suitable for use as medicaments than the compounds of the present invention. This is because certain of the compounds of WO-A-03/101961 are inhibitors of cytochrome P₄₅₀s and this has implications for the metabolism of any pharmacological agent which may be co-administered with these compounds. In contrast, the present inventors have shown that, surprisingly, the compounds of the present invention do not inhibit cytochrome P₄₅₀s. In addition, our preliminary binding experiments have indicated that the sulfide compounds described in WO-A-03/101961 appear to bind human eosinophils with a low off rate, which could lead to an unpredictable duration of action.

WO-A-03/10981 relates to compounds which are of similar structure to the compounds of the present invention except that the substituent at the 3-position of the indole ring system is a phenyl, naphthyl or heteroaryl group with no SO, SO₂ or SO₂NR⁹ linker as with the compounds of general formula (I). Clearly, the inclusion of a linking group is likely to have a substantial effect on the activity of the compound. Furthermore, the substituent at the indole 3-position cannot be an aliphatic group as in the present invention.

WO-A-2004/007451 relates to CRTH2 inhibitors which are similar in structure to the compounds of the present invention in which X is a bond, except that the group equivalent to the R⁸ group of the compounds of general formula (I) is phenyl, naphthyl or a 5-7 membered heteroaromatic group. In fact, all the exemplified compounds have a substituted phenyl group at this position. This is clearly different from the compounds of the present invention where the R⁸ groups are either a bicyclic or tricyclic heteroaromatic ring or an alkyl, alkenyl or alkynyl group. It is particularly surprising that compounds containing alkyl, alkenyl and alkynyl groups have proved to be so active since they differ markedly in structure from the prior art compounds.

In the present specification “C₁-C₆ alkyl” refers to a straight or branched saturated hydrocarbon chain having one to six carbon atoms and optionally substituted with one or more halo substituents or with one or more C₃-C₇ cycloalkyl groups. Examples include methyl, ethyl, n-propyl, isopropyl, t-butyl, n-hexyl, trifluoromethyl, 2-chloroethyl, methylenecyclopropyl, methylenecyclobutyl, methylenecyclobutyl and methylenecyclopentyl.

“C₁-C₄ alkyl” and “C₁-C₁₈ alkyl” have similar meanings except that they contain from one to four and from one to eighteen carbon atoms respectively.

C₃-C₇ cycloalkyl refers to a saturated 3 to 7 membered carbocyclic ring. Examples of such groups include cyclopropyl, cyclobutyl, cyclopentyl and cyclohexyl.

The terms “C₂-C₆ alkenyl” and “C₂-C₆ alkynyl” refer straight or branched hydrocarbon chains having from two to six carbon atoms and containing respectively at least one carbon-carbon double bond or at least one carbon-carbon triple bond. As with alkyl groups they may optionally be substituted with one or more halo substituents or with one or more C₃-C₇ cycloalkyl groups.

In the present specification, “halo” refers to fluoro, chloro, bromo or iodo.

The terms “aromatic moiety” and “aryl” in the context of the present specification refer to an aromatic ring system having from 5 to 14 ring carbon atoms and containing up to three rings. Examples of aromatic moieties are benzene and naphthalene. Aryl groups may be substituted with one or more substituents chosen from halo, C₁-C₆ alkyl, C₁-C₆ alkoxy, a 5-7-membered heterocyclic ring or SO₂R⁹ where R⁹ is as defined above.

The terms “heteroaromatic moiety” and “heteroaryl” refer to an aromatic ring system in which at least one of the ring carbon atoms is replaced by a nitrogen, oxygen or sulfur atom. Examples include single ring systems such as pyridine, pyrimidine, pyrazole, thiophene, oxazole and isoxazole. Other examples include fused ring systems such as quinoline, isoquinoline, quinazoline, benzthiazole, benzoxazole, benzimidazole and indole groups.

Unless stated otherwise a heteroaromatic moiety has from 5 to 14 ring carbon atoms but, for example, “5-7 membered heteroatomatic ring” contains 5 to 7 ring atoms. Bicyclic and tricyclic heteroaryl groups contain respectively two or three fused rings. Bicyclic heteroaryl groups may be, for example, 6,6- or 6-5-ring systems such as those exemplified above.

As with aryl groups, heteroaryl groups may also be substituted with one or more substituents chosen from halo, C₁-C₆ alkyl, C₁-C₆ alkoxy, a 5-7-membered heterocyclic ring or SO₂R⁹ where R⁹ is as defined above.

The term “5 to 7 membered heterocyclic ring” refers to a non-aromatic ring system having from 5 to 7 ring atoms and wherein at least one of the ring carbon atoms is replaced by a nitrogen, oxygen or sulfur atom. Examples include piperidine, morpholine, imidazoline, piperazine and terahydrofuran.

Appropriate pharmaceutically and veterinarily acceptable salts of the compounds of general formulae (I) and (II) include basic addition salts such as sodium, potassium, calcium, aluminium, zinc, magnesium and other metal salts as well as choline, diethanolamine, ethanolamine, ethyl diamine and other well known basic addition salts.

Where appropriate, pharmaceutically or veterinarily acceptable salts may also include salts of organic acids, especially carboxylic acids, including but not limited to acetate, trifluoroacetate, lactate, gluconate, citrate, tartrate, maleate, malate, pantothenate, adipate, alginate, aspartate, benzoate, butyrate, digluconate, cyclopentanate, glucoheptanate, glycerophosphate, oxalate, heptanoate, hexanoate, fumarate, nicotinate, pamoate, pectinate, 3-phenylpropionate, picrate, pivalate, proprionate, tartrate, lactobionate, pivolate, camphorate, undecanoate and succinate, organic sulfonic acids such as methanesulfonate, ethanesulfonate, 2-hydroxyethane sulfonate, camphorsulfonate, 2-naphthalenesulfonate, benzenesulfonate, p-chlorobenzenesulfonate and p-toluenesulfonate; and inorganic acids such as hydrochloride, hydrobromide, hydroiodide, sulfate, bisulfate, hemisulfate, thiocyanate, persulfate, phosphoric and sulfonic acids.

Salts which are not pharmaceutically or veterinarily acceptable may still be valuable as intermediates.

Prodrugs are any covalently bonded compounds which release the active parent drug according to general formula (I) in vivo. Examples of prodrugs include alkyl esters of the compounds of general formula (I), for example the esters of general formula (II) below.

If a chiral centre or another form of isomeric centre is present in a compound of the present invention, all forms of such isomer or isomers, including enantiomers and diastereoisomers, are intended to be covered herein. Compounds of the invention containing a chiral centre may be used as a racemic mixture, an enantiomerically enriched mixture, or the racemic mixture may be separated using well-known techniques and an individual enantiomer may be used alone.

In the compounds of general formula (I), it is preferred that, independently or in any combination:
R¹ is halo or hydrogen;
R² is halo or hydrogen;
R³ is halo or hydrogen;
R⁴ is halo or hydrogen.

In more preferred compounds, R¹, R³ and R⁴ are hydrogen, while R² is halo, particularly fluoro.

In preferred compounds of general formula (I), R⁵ and R⁶ are each independently hydrogen or C₁-C₄ alkyl. However, in more active compounds, at least one, and preferably both of R⁵ and R⁶ are hydrogen.

Compounds of general formula (I) preferably have an R⁷ group chosen from H or C₁-C₆ alkyl; most suitably R⁷ is methyl.

In particularly preferred compounds of general formula (I), n is 2.

When X is a bond, it is preferred that R⁸ is C₁-C₆ alkyl, biphenyl or a bicyclic heteroaryl group, any of which may be substituted with halogen, phenyl, —CO₂R⁹ CON(R⁹)₂ or —SO₂R⁹, where R⁹ is as defined above.

More preferred compounds in which X is a bond include those in which R⁸ is C₁-C₄ alkyl, biphenyl or a bicyclic heteroaryl group, any of which may be substituted with phenyl, —CO₂R⁹ CON(R⁹)₂ or —SO₂R⁹, where R⁹ is H or C₁-C₄ alkyl.

When X is NR⁹, it is preferred that R⁹ is H or methyl and R⁸ is:
phenyl optionally substituted with one or more halo, C₁-C₆alkyl or —O(C₁-C₆ alkyl) groups;
C₁-C₆ alkyl, optionally substituted with aryl; or heteroaryl.

More preferably. when X is NR⁹, R⁸ is phenyl, benzyl or pyridyl, any of which may optionally be substituted with one or more halo, methyl or methoxy groups.

Among the most preferred compounds are the following:
1. [3-(Butane-1-sulfonyl)-5-fluoro-2-methyl-indol-1-yl]-acetic acid
2. 3-(Biphenyl4-sulfonyl)-5-fluoro-2-methyl-indol-1-yl]-acetic acid
3. (3-Carboxymethanesulfonyl-5-fluoro-2-methyl-indol-1-yl)-acetic acid
4. (3-Carbamoylmethanesulfonyl-5-fluoro-2-methyl-indol-1-yl)-acetic acid
5. [5-Fluoro-3-(2-methanesulfonyl-ethanesulfonyl)-2-methyl-indol-1-yl]-acetic acid
6. [3-(Benzothiazole-2-sulfonyl)-5-fluoro-2-methyl-indol-1-yl]-acetic acid
7. [3-(Benzothiazole-2-sulfinyl)-5-fluoro-2-methyl-indol-1-yl]-acetic acid
8. [5-Fluoro-2-methyl-3-(quinoline-2-sulfonyl)-indol-1-yl]-acetic acid
9. [5-Fluoro-2-methyl-3-(quinolin-8-ylsulfonyl)-indol-1-yl]-acetic acid
10. (5-Fluoro-2-methyl-3-phenylmethanesulfonyl-1H-indol-1-yl)-acetic acid
11. [3-(4-Chloro-phenylsulfamoyl)-5-fluoro-2-methyl-indol-1-yl]-acetic acid
12. [3-(3-Chloro-phenylsulfamoyl)-5-fluoro-2-methyl-indol-1-yl]-acetic acid
13. [3-(4-Fluoro-phenylsulfamoyl)-5-fluoro-2-methyl-indol-1-yl]-acetic acid
14. [3-(2-Chloro-phenylsulfamoyl)-5-fluoro-2-methyl-indol-1-yl]-acetic acid
15. (3-Benzylsulfamoyl-5-fluoro-2-methyl-indol-1-yl)-acetic acid
16. [5-Fluoro-3-(2-methoxy-phenylsulfamoyl)-2-methyl-indol-1-yl]-acetic acid
17. [5-Fluoro-3-(4-methoxy-phenylsulfamoyl)-2-methyl-indol-1-yl]-acetic acid
18. (5-Fluoro-2-methyl-3-phenylsulfamoyl-indol-1-yl)-acetic acid
19. [3-(3,4-Dichloro-benzylsulfamoyl)-5-fluoro-2-methyl-indol-1-yl]-acetic acid
20. [5-Fluoro-3-(3-methoxy-phenylsulfamoyl)-2-methyl-indol-1-yl]-acetic acid
21. (5-Fluoro-2-methyl-3-m-tolylsulfamoyl-indol-1-yl)-acetic acid
22. (5-Fluoro-2-methyl-3-p-tolylsulfamoyl-indol-1-yl)-acetic acid
23. [3-(4-Chloro-benzylsulfamoyl)-5-fluoro-2-methyl-indol-1-yl]-acetic acid
24. [3-(Benzyl-methyl-sulfamoyl)-5-fluoro-2-methyl-indol-1-yl]-acetic acid
25. [5-Fluoro-2-methyl-3-(pyridin-3-ylsulfamoyl)-indol-1-yl]-acetic acid; or the C₁-C₆ alkyl, aryl, (CH₂)ₘOC(═O)C₁-C₆alkyl, (CH₂)ₘN(R¹¹)₂, CH((CH₂)ₘO(C═O)R¹²)₂ esters of any of the above; wherein
   m is 1 or 2;
   R¹¹ is hydrogen or methyl;
   R¹² is C₁-C₁₈ alkyl.

In a further aspect of the present invention, there is provided a compound of general formula (II): 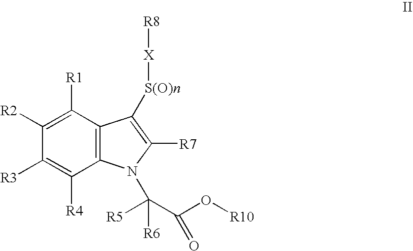
wherein R¹, R², R³, R⁴, R⁵, R⁶, n, X, R⁷ and R⁸ are as defined for general formula (I);
R¹⁰ is C₁-C₆ alkyl, aryl, (CH₂)ₘOC(═O)C₁-C₆alkyl, (CH₂)ₘ(R¹¹)₂, CH((CH₂)ₘO(C═O)R¹²)₂;
   m is 1 or 2;
   R¹¹ is hydrogen or methyl;
   R¹² is C₁-C₁₈ alkyl.

Compounds of general formula (II) are novel and may be used as prodrugs for compounds of general formula (I). When the compound of general formula (II) acts as a prodrug, it is later transformed to the drug by the action of an esterase in the blood or in a tissue of the patient.

Examples of particularly suitable R¹⁰ groups when the compound of general formula (II) is used as a prodrug include:
methyl, ethyl, propyl, phenyl, CH₂OC(═O)tBu, CH₂CH₂N(Me)₂ CH₂CH₂NH₂ or CH(CH₂O(C═O)R¹²)₂ wherein R¹² is as defined above.

Compounds of general formula (I) wherein R¹, R², R³, R⁴, R⁵, R⁶, R⁷ and R⁸ are as defined for general formula (I) and X is a bond, may be prepared from compounds of general formula (Ia), which is a compound of general formula (I) wherein n is 0 and X is a bond, by oxidation with a suitable oxidising agent such as potassium peroxymonosulfate, m-CPBA, hydrogen peroxide or other well known oxidising reagents.

In addition to their use as prodrugs, compounds of formula (II) wherein R¹⁰ is C₁-C₆ alkyl may be used in a process for the preparation of a compound of general formula (I), the process comprising reacting the compound of general formula (II) with a base such as sodium hydroxide or lithium hydroxide. The reaction may take place in an aqueous solvent or an organic solvent or a mixture of the two. A typical solvent used for the reaction is a mixture of tetrahydrofuran and water. The same method may be used to prepare compounds of general formula (Ia) as defined above from compounds of general formula (IIa), which are identical to compounds of general formula (II) except that n is 0.

Compounds of general formula (II) and (IIa) in which X is a bond may be prepared from compounds of general formula (III): 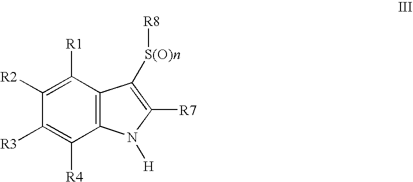
wherein R¹, R², R³, R⁴, R⁷ and R⁸ are as defined for general formula (I) and n is 0, 1 or 2;
by reaction with a compound of general formula (IV):
X—CR⁵R⁶—CO₂R¹⁰ (IV)
wherein R⁵ and R⁶ are as defined for general formula (I), R¹⁰ is as defined for general formula (II) and X is a leaving group in particular a halo group, for example bromo. The reaction is conducted under strongly basic conditions, for example in the presence of excess sodium hydride, and in a polar organic solvent such as dimethylformamide.

Compounds of general formula (IV) are well known and are readily available or can be prepared by methods known to those skilled in the art.

Compounds of general formula (III) wherein R¹, R², R³, R⁴, R⁷ and R⁸ are as defined for general formula (I) and n is 2 can be prepared by reacting a compound of general formula (V): 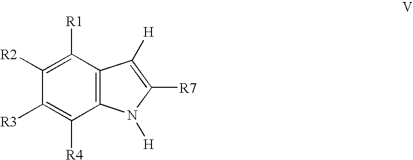
wherein R¹, R², R³, R⁴ and R⁷ are as defined in general formula (I);
with a compound of general formula (VI):
R⁸—SO₂Cl (VI)
wherein R⁸ is as defined in general formula (I).

The reaction is carried out in the presence of a Lewis acid such as indium(III) bromide. The reaction may be conducted in a polar organic solvent, particularly a chlorinated solvent such as 1,2-dichloroethane

Compounds of general formulae (V) and (VI) are well known in the art and are readily available or can be prepared by known methods.

Compounds of general formula (II) in which X is NR⁹ may be prepared from compounds of general formula (VII): 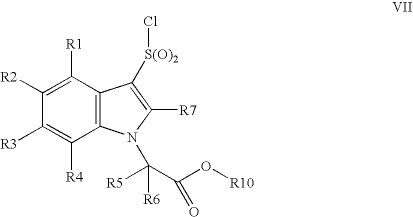
wherein R¹, R², R³, R⁴, R⁵, R⁶ and R⁷ are as defined for general formula (I) and R¹⁰ is as defined in general formula (II) by reaction with a compound of general formula (VIII):
HNR⁸R⁹ (VIII)
wherein R⁸ and R⁹ is as defined above for general formula (I).

The reaction solvent may be a polar organic solvent such as dichloromethane.

Compounds of general formulae (VIII) are well known and are either readily available or can be prepared by methods well known to those skilled in the art.

Compounds of general formula (VII) may be prepared from compounds of general formula (IX) 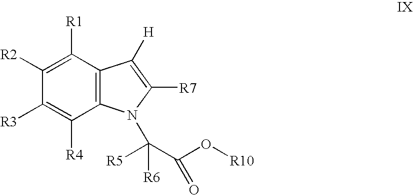
wherein R¹, R² R³, R⁴, R⁵, R⁶, and R⁷ are as defined in general formula (I) and R¹⁰ is as defined for general formula (II);
by reaction with chlorosulfonic acid.

The reaction preferably takes place in a non polar organic solvent.

Compounds of general formula (IX) are well known and are readily available or can be prepared by methods well known to those skilled in the art.

Compounds of general formula (III) wherein R¹, R², R³, R⁴, R⁷ and R⁸ are as defined for general formula (I) and n is 0 can be prepared by reacting a compound of general formula (IX) wherein R¹, R², R³, R⁴ and R⁷ are as defined in general formula (I) and R¹⁰ is as defined for general formula (II) with a compound of general formula (X):
R⁸—SH (x)

wherein R⁸ is as defined in general formula (I).

The reaction is carried out in the presence of iodine and potassium iodide. The reaction may take place in an aqueous or an organic solvent or a mixture of the two. A typical solvent used for the reaction is a mixture such as ethanol and water.

Compounds of general formula (I) are antagonists of PGD₂ at the CRTH2 receptor and compounds of general formula (II) are prodrugs for compounds of general formula (I). Compounds of general formulae (I) and (II) are therefore useful in a method for the treatment of diseases and conditions mediated by PGD₂ at the CRTH2 receptor, the method comprising administering to a patient in need of such treatment a suitable amount of a compound of general formula (I) or (II).

In a third aspect of the invention, there is provided a compound of general formula (I) or (II) for use in medicine, particularly for use in the treatment or prevention of diseases and conditions mediated by PGD₂ at the CRTH2 receptor.

Furthermore, there is also provided the use of a compound of general formula (I) or (II) in the preparation of an agent for the treatment or prevention of diseases and conditions mediated by PGD₂ at the CRTH2 receptor.

As mentioned above, such diseases and conditions include allergic asthma, perennial allergic rhinitis, seasonal allergic rhinitis, atopic dermatitis, contact hypersensitivity (including contact dermatitis), conjunctivitis, especially allergic conjunctivitis, eosinophilic bronchitis, food allergies, eosinophilic gastroenteritis, inflammatory bowel disease, ulcerative colitis and Crohn's disease, mastocytosis and also other PGD₂-mediated diseases, for example autoimmune diseases such as hyper IgE syndrome and systemic lupus erythematus, psoriasis, acne, multiple sclerosis, allograft rejection, reperfusion injury, chronic obstructive pulmonary disease, as well as rheumatoid arthritis, psoriatic arthritis and osteoarthritis.

The compounds of general formula (I) or (II) must be formulated in an appropriate manner depending upon the diseases or conditions they are required to treat.

Therefore, in a further aspect of the invention there is provided a pharmaceutical composition comprising a compound of general formula (I) or (II) together with a pharmaceutical excipient or carrier. Other active materials may also be present, as may be considered appropriate or advisable for the disease or condition being treated or prevented.

The carrier, or, if more than one be present, each of the carriers, must be acceptable in the sense of being compatible with the other ingredients of the formulation and not deleterious to the recipient.

The formulations include those suitable for oral, rectal, nasal, bronchial (inhaled), topical (including eye drops, buccal and sublingual), vaginal or parenteral (including subcutaneous, intramuscular, intravenous and intradermal) administration and may be prepared by any methods well known in the art of pharmacy.

The route of administration will depend upon the condition to be treated but preferred compositions are formulated for oral, nasal, bronchial or topical administration.

The composition may be prepared by bringing into association the above defined active agent with the carrier. In general, the formulations are prepared by uniformly and intimately bringing into association the active agent with liquid carriers or finely divided solid carriers or both, and then if necessary shaping the product. The invention extends to methods for preparing a pharmaceutical composition comprising bringing a compound of general formula (I) or (II) in conjunction or association with a pharmaceutically or veterinarily acceptable carrier or vehicle.

Formulations for oral administration in the present invention may be presented as: discrete units such as capsules, sachets or tablets each containing a predetermined amount of the active agent; as a powder or granules; as a solution or a suspension of the active agent in an aqueous liquid or a non-aqueous liquid; or as an oil-in-water liquid emulsion or a water in oil liquid emulsion; or as a bolus etc.

For compositions for oral administration (e.g. tablets and capsules), the term “acceptable carrier” includes vehicles such as common excipients e.g. binding agents, for example syrup, acacia, gelatin, sorbitol, tragacanth, polyvinylpyrrolidone (Povidone), methylcellulose, ethylcellulose, sodium carboxymethylcellulose, hydroxypropylmethylcellulose, sucrose and starch; fillers and carriers, for example corn starch, gelatin, lactose, sucrose, microcrystalline cellulose, kaolin, mannitol, dicalcium phosphate, sodium chloride and alginic acid; and lubricants such as magnesium stearate, sodium stearate and other metallic stearates, glycerol stearate stearic acid, silicone fluid, talc waxes, oils and colloidal silica. Flavouring agents such as peppermint, oil of wintergreen, cherry flavouring and the like can also be used. It may be desirable to add a colouring agent to make the dosage form readily identifiable. Tablets may also be coated by methods well known in the art.

A tablet may be made by compression or moulding, optionally with one or more accessory ingredients. Compressed tablets may be prepared by compressing in a suitable machine the active agent in a free flowing form such as a powder or granules, optionally mixed with a binder, lubricant, inert diluent, preservative, surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active agent.

Other formulations suitable for oral administration include lozenges comprising the active agent in a flavoured base, usually sucrose and acacia or tragacanth; pastilles comprising the active agent in an inert base such as gelatin and glycerin, or sucrose and acacia; and mouthwashes comprising the active agent in a suitable liquid carrier.

For topical application to the skin, compounds of general formula (I) or (II) may be made up into a cream, ointment, jelly, solution or suspension etc. Cream or ointment formulations that may be used for the drug are conventional formulations well known in the art, for example, as described in standard text books of pharmaceutics such as the British Pharmacopoeia.

Compounds of general formula (I) or (II) may be used for the treatment of the respiratory tract by nasal, bronchial or buccal administration of, for example, aerosols or sprays which can disperse the pharmacological active ingredient in the form of a powder or in the form of drops of a solution or suspension. Pharmaceutical compositions with powder-dispersing properties usually contain, in addition to the active ingredient, a liquid propellant with a boiling point below room temperature and, if desired, adjuncts, such as liquid or solid non-ionic or anionic surfactants and/or diluents. Pharmaceutical compositions in which the pharmacological active ingredient is in solution contain, in addition to this, a suitable propellant, and furthermore, if necessary, an additional solvent and/or a stabiliser. Instead of the propellant, compressed air can also be used, it being possible for this to be produced as required by means of a suitable compression and expansion device.

Parenteral formulations will generally be sterile.

Typically, the dose of the compound will be about 0.01 to 100 mg/kg; so as to maintain the concentration of drug in the plasma at a concentration effective to inhibit PGD₂ at the CRTH2 receptor. The precise amount of a compound of general formula (I) or (II) which is therapeutically effective, and the route by which such compound is best administered, is readily determined by one of ordinary skill in the art by comparing the blood level of the agent to the concentration required to have a therapeutic effect.

Compounds of general formula (I) or (II) may be used in combination with one or more active agents which are useful in the treatment of the diseases and conditions listed above, although these active agents are not necessarily inhibitors of PGD₂ at the CRTH2 receptor.

Therefore, the pharmaceutical composition described above may additionally contain one or more of these active agents.

There is also provided the use of a compound of general formula (I) or (II) in the preparation of an agent for the treatment of diseases and conditions mediated by PGD₂ at the CRTH2 receptor, wherein the agent also comprises an additional active agent useful for the treatment of the same diseases and conditions.

These additional active agents which may have a completely different mode of action include existing therapies for allergic and other inflammatory diseases including:
β2 agonists such as salmeterol;
corticosteroids such as fluticasone;
antihistamines such as loratidine;
leukotriene antagonists such as montelukast;
anti-IgE antibody therapies such as omalizumab;
anti-infectives such as fusidic acid (particularly for the treatment of atopic dermatitis);
anti-fungals such as clotrimazole (particularly for the treatment of atopic dermatitis);
immunosuppressants such as tacrolimus and particularly pimecrolimus in the case of inflammatory skin disease.

CRTH2 antagonists may also be combined with therapies that are in development for inflammatory indications including:
other antagonists of PGD₂ acting at other receptors such as DP antagonists; inhibitors of phoshodiesterase type 4 such as cilonilast;
drugs that modulate cytokine production such as inhibitors of TNFα converting enzyme (TACE);
drugs that modulate the activity of Th2 cytokines IL-4 and IL-5 such as blocking monoclonal antibodies and soluble receptors;
PPAR-γ agonists such as rosiglitazone;
5-lipoxygenase inhibitors such as zileuton.

In yet a further aspect of the invention, there is provided a product comprising a compound of general formula (I) or (II) and one or more of the agents listed above as a combined preparation for simultaneous, separate or sequential use in the treatment of a disease or condition mediated by the action of PGD₂ at the CRTH2 receptor.

The invention will now be described in greater detail with reference to the following non limiting examples and the drawings in which:

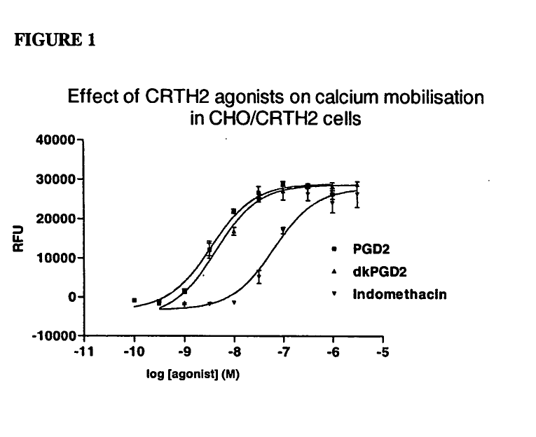

FIG. 1 shows the effects of CRTH2 agonists on calcium mobilisation in CHO/CRTH2 cells.

### EXAMPLE 1

### Synthesis of 3-Sulfonyl Indole Derivatives (Method A)

### 1. Synthesis of 3-(Butane-1-sulfonyl)-5-fluoro-2-methyl-1H-indole

Indium (III) bromide (94.7 mg, 0.267 mmol) was added in one portion to a stirred solution of 5-fluoro-2-methylindole (50 mg, 0.34 mmol) and butanesulfonyl chloride (418 mg, 2.67 mmol) in 1,2-dichloroethane (2 ml) at room temperature. The mixture was subjected to microwave conditions (85° C., 150 W) for 45 minutes, cooled to room temperature and then concentrated in vacuo to leave a brown residue. Purification by flash column chromatography on silica gel eluting with 10% ethyl acetate:hexane to 100% ethyl acetate gave the sulfone (55 mg, 15%) as an off-white solid.

### 2. Synthesis of [3-(Butane-1-sulfonyl)-5-fluoro-2-methyl-indol-1-yl]-acetic acid (Compound 1)

3-(Butane-1-sulfonyl)-5-fluoro-2-methyl-1H-indole (55 mg, 0.204 mmol) in DMF (1 ml) was added dropwise over 1 minute to a stirred suspension of sodium hydride (11 mg, 0.29 mmol; 60% in mineral oil) in DMF (1 ml) at 0° C. The solution was stirred at 0° C. for 45 minutes and then ethyl bromoacetate (0.032 ml, 0.29 mmol) was added dropwise and the resulting mixture stirred at room temperature for 18 hours. The mixture was adjusted to pH 4 with 10% citric acid and then extracted into ethyl acetate (2×10 ml). The combined organic extracts were dried and concentrated in vacuo to leave a residue. The residue was taken up into THF (1 ml) and lithium hydroxide monohydrate (19 mg, 0.464 mmol) in water (1 ml) was then added in one portion at room temperature. The mixture was stirred at room temperature for 3 hours and then the solution adjusted to pH 4 with 10% citric acid. The product was extracted with ethyl acetate and the combined organic extracts were dried and concentrated in vacuo to leave a residue which was triturated with diethyl ether to give the carboxylic acid as an off-white solid (5.4 mg, 8%), δ_{H} (400 MHz, MeOD) 7.57 (1H, dd J 9.8, 2.3 Hz, Ar), 7.43 (1H, dd J 9.1, 4.0 Hz, Ar), 7.04 (1H, td J 9.1, 2.5 Hz, Ar), 4.79 (2H, s, CH₂CO₂H), 3.23-3.19 (2H, m, SO₂CH₂), 2.70 (3H, s, CCH₃), 1.77-1.70 (2H, m, CH₂CH₂CH₂CH₃), 1.47-1.41 (2H, m, CH₂CH₂CH₂CH₃), 0.93 (3H, t J 7.6 Hz, CH₂CH₂CH₂CH₃); Tr=1.38 min, m/z (ES⁺) (M+H)⁺ 308.24. Tr=1.82 min (98%), m/z (ES⁺) (M+H)⁺ 328.20.

Compound 2 was prepared using the same general method as for Compound 1 but with appropriately chosen starting materials.

### Compound 2—3-(Biphenyl4-sulfonyl)-5-fluoro-2-methyl-indol-1-yl]-acetic acid

δ_{H} (400 MHz, MeOD) 8.03 (2H, d, J 8.6 Hz Ar), 7.80 (2H, d, J 8.6 Hz, Ar), 7.77-7.74 (1H, dd, J 9.6, 2.5 Hz, Ar), 7.66-7.64 (2H, dd, J 8.0, 1.3Hz, Ar), 7.49-7.39 (4H, m, Ar), 7.07 (1H, td, J 9.1, 2.5Hz, Ar), 5.07 (2H, s, CH₂), 2.76 (3H, s, CH₃); Tr=1.52 min, m/z (ES⁺) (M+H)⁺ 424.1.

### EXAMPLE 2

### Synthesis of 3-Sulfonyl Indole Derivatives (Method B)

### 1. 2-Methylsulfanyl-ethanethiol

A solution of methyl iodide (10 ml, 22.82 g, 0.161 mol) in acetone (50 mi) was added dropwise to a stirred suspension of ethane dithiol (11.24 ml, 12.62 g, 0.134 mol) and potassium carbonate (37.04 g, 0.268 mol) in acetone (150 ml). The resulting mixture was stirred at room temperature for 4 hours. Water (150 ml) was added, and the mixture stirred for a further 15 minutes. The reaction mixture was extracted with dichloromethane (3×200 ml), the organic washings combined, dried over sodium sulfate and evaporated (maintaining pressure above 200 mbar to ensure no co-evaporation of product) to give 2-methylsulfanyl-ethanethiol. LC/MS showed <5% starting material and a 2:1 mixture of mono and bis-methylated material. The material was used in the next step with no further purification.

### 2. [5-Fluoro-2-methyl-3-(2-methylsulfanyl-ethylsulfanyl)-indol-1-yl]-acetic acid ethyl ester

To a stirred solution of (5-fluoro-2-methyl-indol-1-yl)-acetic acid ethyl ester (1.20 g, 5.10 mmol) and 2-methylsulfanyl-ethanethiol (1.04 g, 6.12 mmol) in 1:1 EtOH:H₂O (40 ml) at room temperature was added iodine (1.29 g, 5.10 mmol) and potassium iodide (0.847 g, 5.10 mmol). The mixture was heated to 100° C. and stirred for 2 hours, then stirred at room temperature for 16 hours. The reaction mixture was quenched by the careful addition of saturated NaHCO_{3 (aq)}, then extracted with DCM (2×50 ml). The organic washings were combined, washed with saturated sodium thiosulfate (2×70 ml), dried over magnesium sulfate and evaporated to give an oil. The crude oil was purified by chromatography (3×12cm column; 4:1 hexane:EtOAc as eluant) to give the ester (1.17 g, 67%). δ_{H} (400 MHz, CDCl₃) 7.36 (1H, dd J 9.2, 2.5 Hz, Ar), 7.11 (1H, dd J 8.8, 4.1 Hz, Ar), 7.05 (1H, td J 9.2, 2.3 Hz, Ar), 4.79 (2H, s, CH₂CO₂Et), 4.21 (2H, q J 7.2 Hz, CO₂CH₂CH₃), 2.86-2.79 (2H, m, CH₂CH₂), 2.59-2.55 (2H, m, CH₂CH₂), 2.50 (3H, s, SCH₃), 2.04 (3H, s, CCH₃), 1.25 (3H, t J 7.2 Hz, CO₂CH₂CH₃).

### 3. [5-Fluoro-3-(2-methanesulfonyl-ethanesulfonyl)-2-methyl-indol-1-yl]-acetic acid ethyl ester

Oxone (8.43 g, 13.7 mmol) was added to a stirred solution of [5-fluoro-2-methyl-3-(2-methylsulfanyl-ethylsulfanyl)-indol-1-yl]-acetic acid ethyl ester (1.17 g, 3.43 mmol) in 4:1 1,4-dioxane:H₂O at room temperature. After 30 minutes the reaction mixture was quenched by the careful addition of saturated sodium bicarbonate (50 ml; care—effervescence), then extracted with DCM (2×100 ml). The organic washings were combined and washed with brine (2×100 ml). Aqueous washings were then back-extracted with DCM (100 ml). All organic layers were combined, dried over magnesium sulfate and evaporated to give a pale green crystalline solid. The solid was suspended in DCM and collected via filtration to give the ester (1.06 g, 76%). δ_{H} (400 MHz, CDCl₃) 7.66 (1H, dd J 9.i, 2.5 Hz, Ar), 7.22 (1H, dd J 9.0, 4.0 Hz, Ar), 7.08 (1H, td J 8.9, 2.5 Hz, Ar), 4.86 (2H, s, CH₂CO₂Et), 4.26 (2H, q J 7.1 Hz, CO₂CH₂CH₃), 3.61-3.57 (2H, m, CH₂CH₂), 3.48-3.44 (2H, m, CH₂CH₂), 2.99 (3H, s, SCH₃), 2.71 (3H, s, CH₃), 1.30 (3H, t J 7.1 Hz, CO₂CH₂CH₃).

### 4. Compound 5—[[5-Fluoro-3-(2-methanesulfonyl-ethanesuffonyl)-2-methyl-indol-1-yl]-acetic acid

Lithium hydroxide monohydrate (132 mg, 3.14 mmol) was added in one portion to a stirred solution of [5-fluoro-3-(2-methanesulfonyl-ethanesulfonyl)-2-methyl-indol-1-yl]-acetic acid ethyl ester (1.06 g, 2.61 mmol) in THF:water (5:1; 15 ml) and the resulting mixture stirred at room temperature for 2 h. The mixture was concentrated in vacuo to leave a residue which was partitioned between ethyl acetate and 10% citric acid. The organic layer was separated and the aqueous solution extracted with ethyl acetate (3×100 ml). The combined organic extracts were dried and concentrated in vacuo to leave an off-white solid. The solid was then triturated with dichloromethane to give the carboxylic acid as an off-white solid, (438 mg, 44%), δ_{H} (400 MHz, d6-Acetone) 7.58-7.63 (2H, m, Ar) 7.09 (1H, td J 9.2, 2.6 Hz, Ar), 5.22 (2H, s, CH₂CO₂H), 3.60-3.66 (2H, m, SO₂CH₂CH₂), 3.45 (2H, m, SO₂CH₂CH₂), 3.02 (3H, s, SO₂CH₃), 2.75 (3H, s, CH₃); Tr=1.08 min (98%), m/z (ES⁺) (M+H)⁺ 378.16.

Compounds 3 and 4 were prepared by a similar route using appropriate starting materials.

### Compound 3—(3-Carboxymethanesulfonyl-5-fluoro-2-methyl-indol-1-yl)-acetic acid

Tr=1.16 min, m/z (ES⁺) (M+H)⁺ 330.10.

### Compound 4—(3-Carbamoylmethanesulfony-5-fluoro-2-methyl-indol-1-yl)-acetic acid

Tr=1.50 min, m/z (ES⁺) (M+H)⁺ 329.17.

### EXAMPLE 3

### Synthesis of 3-Sulfonyl Indole Derivatives (Method B2)

A similar method to that set out in step 2 of Example 2 above was used to synthesise the following intermediates. However, hydrolysis to the acid took place before oxidation to give the sulfone or sulfoxide derivative.

### [5-Fluoro-2-methyl-3-(quinolin-8-ylsulfanyl)-indol-1-yl]-acetic acid

δ_{H} (400 MHz, MeOD) 8.95-8.94 (1H, m, Ar), 8.36 (1H, dd J 8.3, 1.7 Hz, Ar), 7.64-7.60 (2H, m, Ar), 7.45 (1H, dd J 8.8,4.2 Hz, Ar), 7.29 (1H, t J 7.8 Hz, Ar), 7.09 (1H, dd J 9.2, 2.6 Hz, Ar), 7.00 (1H, td J 9.2, 2.6 Hz, Ar), 6.85 (1H, app d J 7.3 Hz, Ar), 5.14 (2H, s, CH₂CO₂H), 2.52 (3H, s, CCH₃); Tr=1.30 min, m/z (ES⁺) (M+H)⁺ 367.39.

### [5-Fluoro-2-methyl-3-(quinolin-2-ylsulfanyl)-indol-1-yl]-acetic acid

δ_{H} H (400 MHz, MeOD) 8.01 (1H, d J 8.6 Hz, Ar), 7.93 (1H, d J 7.8 Hz, Ar), 7.82 (1H, d J 8.1 Hz, Ar), 7.76 (1H, app td J 7.1, 1.4 Hz, Ar), 7.53 (1H, app td J 7.0, 1.1 Hz, Ar), 7.47 (1H, dd J 9.1, 4.2 Hz, Ar), 7.16 (1H, dd J 9.0, 2.4 Hz, Ar), 7.03 (1H, td J 9.2, 2.6 Hz, Ar), 6.87 (1H, d J 8.8 Hz, Ar), 5.14 (2H, s, CH₂CO₂H), 2.55 (3H, s, CCH₃); Tr=1.37 min, m/z (ES⁺) (M+H)⁺ 367.24.

### [3-(Benzothiazol-2-ylsulfanyl)-5-fluoro-2-methyl-indol-1-yl]-acetic acid

δ_{H} (400 MHz, MeOD) 7.81 (1H d J 8.3 Hz, Ar), 7.71 (1H, d J 7.8 Hz, Ar), 7.50-7.43 (2H, m, Ar), 7.31-7.24 (2H, m, Ar), 7.06 (1H td J 9.0, 2.4 Hz, Ar), 5.15 (2H, s, CH₂CO₂H), 2.60 (3H, s, CCH₃); Tr=1.49 min, m/z (ES⁺) (M+H)⁺ 373.34.

### (3-Benzylsulfanyl-5-fluoro-2-methyl-indol-1-yl)-acetic acid

δ_{H} (250 MHz, d₆-DMSO) 7.46 (1H, dd J 8.8, 4.3 Hz, Ar), 7.21-7.18 (3H, m, Ar), 7.14 (1H, dd J 9.5, 2.5 Hz, Ar), 7.01-6.92 (3H, m, Ar), 4.99 (2H, s, CH₂CO₂H), 3.75 (2H, s, ArCH₂), 2.01 (3H, s, CH₃); Tr=1.56 min (100%) m/z (ES⁺) (M+H)⁺ 330.16.

These intermediates can then be oxidised to give compounds of general formula (I) where n is 1 or 2 using the following method.

### 1. Compound 6—[3-(Benzothiazole-2-sulfonyl)-5-fluoro-2-methyl-indol-1-yl]-acetic acid and Compound 7—[3-(Benzothiazole-2-sulfinyl)-5-fluoro-2-methyl-indol-1-yl]-acetic acid

Potassium peroxymonosulfate (131.0 mg, 214 mmol) was added in one portion to a stirred solution of the [3-(benzothiazol-2-ylsulfanyl)-5-fluoro-2-methyl-indol-1-yl]-acetic acid, 20.0 mg, 53.6 mmol) in 1, 4-dioxane:water (0.3 ml; 4:1) at room temperature. The mixture was stirred at room temperature for 18 h and then a 25 saturated solution of sodium bicarbonate (5 ml) was added. The product was extracted with ethyl acetate (3×2 ml) and the combined organic extracts were washed with brine, dried and concentrated in vacuo to leave a solid which was purified by preparative HPLC to give the sulfone, Compound 6 (10.0 mg, 46%) as an off-white solid, δ_{H} (400 MHz, MeOD) 8.11 (2H, obs dd J 7.9, 2.8 Hz, Ar), 7.79 (1H, dd J 9.6, 2.5 Hz, Ar), 7.65-7.57 (2H, m, Ar), 7.43 (1H, dd J 8.8, 4.3 Hz, Ar), 7.06 (1H, td J 9.1, 2.5 Hz, Ar), 4.76 (2H, s, CH₂CO₂H), 2.85 (3H, s, CCH₃); Tr=1.44 min (100%), m/z (ES⁺) (M+H)⁺ 405.21, and the sulfoxide, Compound 7 (3.2 mg, 15%) as an off-white solid, δ_{H} (400 MHz, MeOD) 8.16 (1H, app d J 9.1 Hz, Ar), 8.01 (1H, d J 8.1 Hz, Ar), 7.62-7.54 (2H, m, Ar), 7.47 (1H, dd J9.1, 4.0 Hz, Ar), 7.23 (1H, dd J 9.6, 2.5 Hz, Ar), 7.02 (1H, td J 9.1, 2.0 Hz, Ar), 5.10 (2H, s, CH₂CO₂H), 2.78 (3H, s, CCH₃); Tr=1.34 min (100%), m/z (ES⁺) (M+H)⁺ 389.09.

Compounds 8 to 10 were prepared using the same general method as for Compounds 6 and 7, but with appropriately chosen starting materials.

### Compound 8—[5-Fluoro-2-methyl-3-(quinoline-2-sulfonyl)-indol-1-yl]-acetic acid

δ_{H} (400 MHz, MeOD) 8.57 (1H, d J 8.6 Hz, Ar), 8.20 (1H, d J 8.6 Hz, Ar), 8.13 (1H, d J 8.6 Hz, Ar), 8.02 (1H, d J 8.1 Hz, Ar), 7.89-7.82 (2H, m, Ar), 7.73 (1H, app t J 8.1 Hz, Ar), 7.42 (1H, dd J 8.8, 4.3 Hz, Ar), 7.05 (1H, td J 9.1, 2.5 Hz, Ar), 5.08 (2H, s, CH₂CO₂H), 2.86 (3H, s, CCH₃); Tr=1.39 min (92%), m/z (ES⁺) M+H)⁺ 399.26.

### Compound 9—[5-Fluoro-2-methyl-3-(quinolin-8-ylsulfonyl)-indol-1-yl]-acetic acid

δ_{H} (400 MHz, MeOD) 8.89 (1H, app d J 4.3 Hz, Ar), 8.71 (1H, dd J 7.3 Hz, Ar), 8.34 (1H, app d J 8.3 Hz, Ar), 8.20 (1H, app d J 8.3 Hz, Ar), 7.80 (1H, t J 8.1 Hz, Ar), 7.58 (1H, dd J 10.1, 2.5 Hz, Ar), 7.53 (1H, dd J 8.3, 4.3 Hz, Ar), 7.34 (1H, dd J 8.8, 4.3 Hz, Ar), 6.95 (1H, td J 9.1, 2.5 Hz, Ar), 5.02 (2H, s, CH₂CO₂H), 2.97 (3H, s, CCH₃); Tr=1.78 min (100%), m/z (ES⁺) (M+H)⁺ 399.29.

### Compound 10—(5-Fluoro-2-methyl-3-phenylmethanesulfonyl-1H-indol-1-yl)-acetic acid

δ_{H} (250 MHz, d₆-DMSO) 7.61 (1H, dd J 9.0, 4.5 Hz, Ar), 7.35 (1H, dd J 9.8, 2.5 Hz, Ar), 7.30-7.19 (3H, m, Ar), 7.10 (1H, td J 9.1, 2.6 Hz, Ar), 7.02 (2H, m, Ar), 5.10 (2H, s, CH₂CO₂H), 4.51 (2H, s, ArCH₂), 2.06 (3H, s, CH₃); Tr=1.30 min (100%) 30 m/z (ES⁺) (M+H)⁺ 362.13.

### EXAMPLE 4

### Synthesis of 3-Sulfamoyl Indole Derivatives (Method C)

The method described below is employed for compounds of general formula (I) in which X is NR⁹.

### 1. [3-(4-Chloro-phenylsulfamoyl)-5-fluoro-2-methyl-indol-1-yl]-acetic acid ethyl ester

Chlorosulfonic acid (0.042 ml, 0.63 mmol) was added dropwise over 1 min to a stirred solution of (5-fluoro-2-methyl-indol-1-yl)-acetic acid ethyl ester (100 mg, 0.43 mmol) in ether (1 ml) at 0° C. The solution was stirred at 0° C. for 10 min and then concentrated in vacuo to leave a residue which was azeotroped with dichloromethane (2×2 ml). The residue was taken up in dichloromethane and then N,N-diisopropyl ethylamine (0.075 ml, 0.43 mmol) and 4-chloroaniline (53.4 mg, 0.42 mmol)) were added. The resulting mixture was stirred at room temperature for 40 min and then concentrated in vacuo to leave a residue which was partitioned between ethyl acetate (5 ml) and water (5 ml). The organic layer was then separated, washed with a saturated solution of sodium hydroxide (20 ml), dried and concentrated in vacuo to leave a residue which was purified by flash column chromatography (Flashmaster) on silica gel eluting with 15% ethyl acetate:heptane to give the sulfonamide (6 mg, 3%) as an off-white solid, δ_{H} (400 MHz, CDCl₃) 7.63 (1H, dd J 9.5, 2.4 Hz, Ar), 7.18-7.12 (3H, m, Ar), 7.05-6.99 (1H, m, Ar), 6.96-6.90 (2H, m, Ar), 6.55 (1H, s, NH), 4.73 (2H, s, NCH₂), 4.20 (2H, q J 7.3 Hz, OCH₂CH₃), 2.33 (3H, s, CCH₃), 1.22 (3H, t J 7.3 Hz, OCH₂CH₃); Tr=1.57 min (100%), m/z (ES⁺) (M+H)⁺ 425.

### 2. Compound 11—[3-(4-Chloro-phenylsulfamoyl)-5-fluoro-2-methyl-indol-1-yl]-acetic acid

Lithium hydroxide monohydrate (7.0 mg, 0.17 mmol) in water (2 ml) was added in one portion to a stirred solution of [3-(4-chloro-phenylsulfamoyl)-5-fluoro-2-methyl-indol-1-yl]-acetic acid ethyl ester (6 mg, 0.014 mmol) in tetrahydrofuran (2 ml). The resulting mixture was stirred at room temperature for 3 h and then the pH of the mixture was adjusted to pH 1 with IM hydrochloric acid. The product was extracted with ethyl acetate (2×10 ml) and the combined organic extracts were then dried and concentrated in vacuo to give the carboxylic acid (4.3 mg, 77%) as an off-white solid, δ_{H} (400 MHz, CDCl₃) 8.74 (1H, s, NH), 7.70 (1H, dd J 9.5, 2.6 Hz, Ar), 7.13-7.06 (3H, m, Ar),6.99-6.92 (3H, m, Ar), 4.67 (2H, s, NCH₂), 2.41 (3H, s, CH₃); Tr=1.84 min (91%), m/z (ES⁺) (M+H)⁺ 397.

Compounds 12 to 25 were prepared using the same general method but with appropriately chosen starting materials.

### Compound 12—[3-(3-Chloro-phenylsulfamoyl)-5-fluoro-2-methyl-indol-1-yl]-acetic acid

δ_{H} (400 MHz, CDCl₃) 7.63 (1H, dd J 9.3, 2.6 Hz, Ar), 7.17-7.14 (1H, m, Ar), 7.10-6.98 (5H, m, Ar, NH), 6.86-6.84 (1H, m, Ar), 4.73 (2H, s, NCH₂), 2.46 (3H, s, CH₃); Tr=1.84 min (100%), m/z (ES⁺) (M+H)⁺ 397.

### Compound 13—[3-(4-Fluoro-phenylsulfamoyl)-5-fluoro-2-methyl-indol-1-yl]-acetic acid

δ_{H} (400 MHz, CDCl₃) 8.45(1H, s, NH), 7.66(1H, dd J9.7, 2.3Hz, Ar), 7.11 (1H, dd J 9, 4.2Hz, Ar), 6.97-6.90 (3H, m, Ar), 6.81-6.77 (2H, m, Ar), 4.64 (2H, s, NCH₂), 2.29 (3H, s, CH₃); Tr=1.79 min (99%), m/z (ES⁺) (M+H)⁺ 381.

### Compound 14—[3-(2-Chloro-phenylsulfamoyl)-5-fluoro-2-methyl-indol-1-yl]-acetic acid

δ_{H} (400 MHz, CDCl₃) 7.69 (1H, s, NH), 7.58-7.49 (2H, m, Ar), 7.23-7.13 (3H, m, Ar), 7.03-6.93 (2H, m, Ar), 4.70 (2H, s, NCH₂), 2.44 (3H, s, CH₃); Tr=1.83 (100%), m/z (ES⁺) (M+H)⁺ 397.

### Compound 15- (3-Benzylsulfamoyl-5-fluoro-2-methyl-indol-1-yl)-acetic acid

δ_{H} (400 MHz, d₆-DMSO) 7.99 (1H, t J 6.3 Hz, Ar), 7.58 (2H, m, Ar), 7.21 (4H, m, Ar), 7.09 (1H, td J 9.23, 2.65 Hz, Ar), 5.11 (2H, s, CH₂CO₂H), 3.92 (2H, d J 6.31 Hz, NCH₂) 2.56 (3H, s, CH₃), Tr=1.31 min (100%), m/z (ES⁺) (M+H)⁺ 377.25.

### Compound 16—[5-Fluoro-3-(2-methoxy-phenylsulfamoyl)-2-methyl-indol-1-yl]-acetic acid

δ_{H} (400 MHz, d₆-DMSO) 9.18 (1H, s, SO₂NH), 7.52 (1H, dd J 9.00, 4.4 Hz, Ar), 7.47 (1H, dd J 10.2, 2.6 Hz, Ar), 7.23 (1H, dd J 7.9, 1.6 Hz, Ar), 7.09-7.01 (2H, m, Ar), 6.84 (1H, t J 7.7 Hz, Ar), 6.77 (1H, d J 7.2 Hz, Ar), 5.05 (2H, s, CH₂CO₂H), 3.24 (3H, OCH₃), 2.30 (3H, s, CH₃); Tr=1.31 min (100%), m/z (ES⁺) (M+H)⁺ 393.25.

### Compound 17—[5-Fluoro-3-(4-methoxy-phenylsulfamoyl)-2-methyl-indol-1-yl]-acetic acid

δ_{H} (400 MHz, d₆-DMSO) 9.62 (1H, s, SO₂NH), 7.49 (1H, dd J 10.3, 2.6 Hz, Ar), 7.36 (1H, dd J 9.0, 4.6 Hz, Ar), 6.98 (1H, dd J 9.2, 2.8 Hz, Ar), 6.93 (2H, d J 9.1 Hz, Ar), 6.74 (2H, d J 9.1 Hz, Ar), 4.45 (2H, s, CH₂CO₂H), 3.64 (3H, s, OCH₃), 2.36 (3H, s, CH₃); Tr=1.27 min (100%), m/z (ES⁺) (M+H)⁺ 393.26.

### Compound 18—(5-Fluoro-2-methyl-3-phenylsulfamoyl-indol-1-yl)-acetic acid

δ_{H} (400 MHz, d₆-DMSO) 10.15 (1H, s, SO₂NH), 7.60 (1H, dd J 10.1, 2.6 Hz, Ar), 7.53 (1H, dd J 9.1, 4.5 Hz, Ar), 7.17 (2H, m, Ar), 7.07 (1H, dd J 9.1, 2.6 Hz, Ar), 7.03 (2H, m, Ar), 6.96 (1H, t J 7.3 Hz, Ar), 5.03 (2H, s, CH₂CO₂H), 2.48 (3H, s, CH₃); Tr=1.28 min (96%), m/z (ES⁺) (M+H)⁺ 363.25.

### Compound 19—[3-(3,4-Dichloro-benzylsulfamoyl)-5-fluoro-2-methyl-indol-1-yl]-acetic acid

δ_{H} (400 MHz, d₆-DMSO) 7.92 (1H, bs, SO₂NH), 7.47 (1H, dd J 10.3, 2.6 Hz, Ar), 7.40 (1H, d J 8.3 Hz, Ar), 7.37 (1H, d J 1.7 Hz, Ar), 7.31 (1H, dd J 9.0, 4.6 Hz, Ar), 7.13 (1H, dd J 8.2, 2.0 Hz, Ar), 6.96 (1H, td J 9.3, 2.7 Hz, Ar), 4.32 (2H, s, CH₂CO₂H), 3.93 (2H, s, NCH₂), 2.49 (3H, s, CH₃); Tr=1.43 min (97%), m/z (ES⁺) (M+H)⁺ 445.15.

### Compound 20—[5-Fluoro-3-(3-methoxy-phenylsulfamoyl)-2-methyl-indol-1-yl]-acetic acid

δ_{H} (400 MHz, d₆-DMSO) 10.22 (1H, s, SO₂NH), 7.60 (1H, dd J 9.9, 2.5 Hz, Ar), 7.50 (1H, dd J 9.1, 4.1 Hz, Ar), 7.05 (2H, m, Ar), 6.61 (2H, m, Ar), 6.50 (1H, d 8.7 Hz, Ar), 4.88 (2H, s, CH₂CO₂H), 3.60 (3H, s, OCH₃), 2.54 (3H, s, CH₃); Tr=1.28 min (100%), m/z (ES⁺) (M+H)⁺ 393.28.

### Compound 21—(5-Fluoro-2-methyl-3-m-tolylsulfamoyl-indol-1-yl)-acetic acid

δ_{H} (400 MHz, d₆-DMSO) 10.11 (1H, s, SO₂NH), 7.61 (1H, dd J 10.1, 2.6 Hz, Ar), 7.55 (1H, dd J 9.1, 4.5 Hz, Ar), 7.08 (1H, dd J 9.2, 2.6 Hz, Ar), 7.03 (1H, d J 7.8, Ar), 6.85-6.81 (2H, m, Ar), 6.76 (1H, d J 7.6 Hz, Ar), 5.08 (2H, s, CH₂CO₂H), 2.50 (3H, s, CH₃),2.15 (3H, s, ArCH₃), Tr=1.33 min (100%), m/z (ES⁺) (M+H)⁺ 377.24.

### Compound 22—(5-Fluoro-2-methyl-3-p-tolylsulfamoyl-indol-1-yl)-acetic acid

δ_{H} (400 MHz, d₆-DMSO) 9.90 (1H, bs, SO₂NH), 7.55 (1H, dd J 10.2, 2.6 Hz, Ar), 7.32 (1H, dd J 9.1, 4.6, Ar), 6.94 (5H, m, Ar), 4.31 (2H, s, CH₂CO₂H), 2.45 (3H, s, CH₃), 2.15 (3H, s, CH₃), Tr=1.33 min (100%), m/z (ES⁺) (M+H)⁺ 377.25.

### Compound 23—[3-(4-Chloro-benzylsulfamoyl)-5-fluoro-2-methyl-indol-1-yl]-acetic acid

δ_{H} (400 MHz, d₆-DMSO) 8.04 (1H, t J 5.9 Hz, NH), 7.58-7.51 (2H, m, Ar), 7.25 (2H, d J 8.6 Hz, Ar), 7.18 (2H, d J 8.6 Hz, Ar), 7.07 (1H, td J 9.5, 2.7 Hz, Ar), 5.07 (2H, s, CH₂CO₂H), 3.93 (2H, d J 6.3 Hz, NCH₂) 2.56 (3H, s, CH₃); Tr=1.38 min (91%), m/z (ES⁺) (M+H)⁺ 411.07.

### Compound 24—[3-(Benzyl-methyl-sulfamoyl)-5-fluoro-2-methyl-indol-1-yl]-acetic acid

δ_{H} (400 MHz, d₆-DMSO) 7.52 (1H, dd J 10.0, 2.6 Hz, Ar), 7.46 (1H, dd J 8.9, 4.4 Hz, Ar), 7.39-7.28 (5H, m, Ar), 7.03 (1H, td J 9.4, 2.8 Hz, Ar), 4.46 (2H, s, CH₂CO₂H), 4.10 (2H, s, NCH₂), 2.60 (3H, s, NCH₃), 2.48 (3H, s, CH₃); Tr=1.43 min (100%), m/z (ES⁺) (M+H)⁺ 391.15.

### Compound 25—[5-Fluoro-2-methyl-3-(pyridin-3-ylsulfamoyl)-indol-1-yl]-acetic acid

δ_{H} (400 MH, d₆-DMSO) 10.42 (1H, s, SO₂NH), 8.25 (1H, d J 2.6 Hz, Ar), 8.19 (1H, d, 3.3 Hz, Ar), 7.59-7.55 (2H, m, Ar), 7.40 (1H, d J 8.3Hz, Ar), 7.24-7.21 (2H, m, Ar), 5.10 (2H, s, CH₂CO₂H), 2.49 (3H, s, CH₃); Tr=0.96 min (100%), m/z (ES⁺) (M+H)⁺ 364.1.

### EXAMPLE 5

### Measurement of CRTH2 Antagonist Activity

Materials and Methods

Materials

Calcium-3 dye was purchased from Molecular Devices (Wokingham, UK). Monopoly resolving medium was obtained from Dainippon Pharmaceuticals (Osaka, Japan). Macs anti-CD16 microbeads were from Miltenyi biotec (Bisley, Surrey). ChemoTx plates were purchased from Neuroprobe (Gaithesburg, Md.). Poly-D-lysine coated 96-well plates were obtained from Greiner (Gloucestershire, UK). [³H]PGD₂ was from Amersham Biosciences (Buckinghamshire, UK). [³H]SQ29548 was purchased from Perkin Elmer Life Sciences (Buckinghamshire, UK). All other reagents were obtained from Sigma-Aldrich (Dorset, UK), unless otherwise stated.

Methods

Cell Culture

Chinese Hamster Ovary cells were transfected with CRTH2 or DP receptors (CHO/CRTH2 and CHO/DP) and were maintained in culture in a humidified atmosphere at 37° C. (5% CO₂) in Minimum Essential Medium (MEM) supplemented with 10% foetal bovine serum, 2 mM glutamine, and 1 mg ml⁻¹ active G418. The cells were passaged every 2-3 days. For radioligand binding assay, cells were prepared in triple-layer flasks or in 175 cm² square flasks (for membrane preparation). For calcium mobilisation assay, cells were grown in a 96 well plate 24 h prior to the assay at a density of 80,000 cells per well.

Preparation of Cell Membranes

Membranes were prepared either from CHO/CRTH2 and CHO/DP cells, or from platelets (as a source of TP receptors). CHO cells grown to confluency were washed with PBS and detached using a Versene solution (15 ml per flask). When the cells were grown in 175 cm² square flask, they were collected by scrapping in PBS. The cell suspensions were centrifuged (1,700 rpm, 10 min, 4° C.) and resuspended in 15 ml of buffer (1×HBSS, supplemented with 10 mM HEPES, pH 7.3). Cell suspensions were then homogenised using an Ultra Turrax at setting 4-6 for 20 s. The homogenate was centrifuged at 1,700 rpm for 10 min and the supernatant was collected and centrifuged at 20,000 rpm for 1 h at 4° C. The resulting pellet was resuspended in buffer and stored at −80° C. in aliquots of 200-500 μl. The protein concentration was determined by the method of Bradford (1976), using bovine serum albumin as standard. The platelets were washed by centrifugation at 600×g for 10 min and resuspended in ice-cold assay buffer (10 mM Tris-HCl, pH 7.4, 5 mM Glucose, 120 mM NaCl, 10 μM indomethacin) and directly centrifuged at 20,000 rpm for 30 min at 4° C. The resulting pellet was treated as described above.

Radioligand Binding Assays

[3H]PGD₂ (160 Ci/mmol) binding experiments were performed on membranes prepared as described above. Assays were performed in a final volume of 100 μl of buffer (1×HBSS/HEPES 10 mM, pH 7.3). Cell membranes (15 μg). Cell membranes 15 mg were preincubated at room temperature with varying concentration of competing ligand for 15 min. [3H]PGD₂ (mol, final concentration) was then added and the incubation continued for a further one hour at room temperature. The reaction was terminated by the addition of 200 μl ice-cold assay buffer to each well, followed by rapid filtration through Whatman GF/B glass fibre filters using a Unifilter Cell harvester (PerkinElmer Life Sciences) and six washes of 300 μl of ice-cold buffer. The Unifilter plates were dried at room temperature for at least 1 h and the radioactivity retained on the filters was determined on a Beta Trilux counter (PerkinElmer Life Sciences), following addition of 40 μl of Optiphase Hi-Safe 3 (Wallac) liquid scintillation. Non specific binding was defined in the presence of 10 AM unlabelled PGD₂. Assays were performed in duplicate.

The results of the radioligand binding experiments to the CRTH2 and DP receptors are shown in Table 1.

The results shown in Table 1 demonstrate that for compounds of general formula (I) have high affinity for the CRTH2 receptor. In those cases where a comparison was made, the affinity of the compounds of general formula (I) is much higher for the CRTH2 receptor than for DP receptor.

TABLE 1
Radioligand binding data (Ki on
CRTH2 Receptor and DP Receptor).

| | CRTH2 Binding | DP Binding |
|---|---|---|
| Compound | Ki nM | Ki μM |
| 1 | 192 | >10 |
| 2 | 75 | >10 |
| 3 | 2000 | ND |
| 4 | 2300 | ND |
| 5 | 89 | >10 |
| 6 | 209 | ND |
| 7 | 54 | ND |
| 8 | 249 | ND |
| 9 | 254 | >10 |
| 10 | 6 | ND |
| 11 | 51 | >10 |
| 12 | 45 | >10 |
| 13 | 182 | ND |
| 14 | 225 | ND |
| 15 | 278 | >10 |
| 16 | 771 | >10 |
| 17 | 1450 | >10 |
| 18 | 236 | >10 |
| 19 | 181 | >10 |
| 20 | 531 | >10 |
| 21 | 176 | >10 |
| 22 | 1240 | >10 |
| 23 | 164 | >10 |
| 24 | 119 | >10 |
| 25 | 4250 | >10 |

The TP receptor radioligand binding was done on membranes prepared from platelets. 15-40 μg of protein were pre-incubated with varying concentrations of competing ligand for 15 min at room temperature in assay buffer (10 mM Tris-HCl, pH 7.4, 5 mM glucose, 120 mM NaCl, 10 μM indomethacin). [³ H]SQ29548 (38 Ci/mmol, 10 nM final concentration) was then added and the incubation continued for a further 30 min at room temperature. The reaction was terminated by the addition of 200 μl ice-cold assay buffer to each well, followed by rapid filtration through Whatman GF/C glass fibre filters using a Unifilter Cell harvester (PerkinElmer Life Sciences) followed with six washes of 300 μl of ice-cold buffer.

The radioactivity was determined as described above.

All of the compounds studied in this assay bound to the TP receptor with low affinity (Ki>10 μM).

Compounds of general formula (I) bound to CRTH2 receptor expressed in CHO cells with a range of affinity varying from very high to moderate. In fact the Ki values determined in competition versus [³H]PGD₂ varied from 500 pM to 1 μM. Compounds of general formula (I) had no activity (or very weak activity) at the DP and TP receptors. The binding selectivity of the compounds of general formula (I) for CRTH2 receptor was greater than 200 fold for CRTH2 receptor, compared to DP and TP receptors.

Calcium Mobilisation Assay

Cells were seeded onto poly-D-lysine coated 96-well plates at a density of 80,000 cells per well and incubated at 37° C. overnight to allow the cells to adhere. Cells were washed twice with HBSS and incubated for 1 h at 37° C. in 100 μl HBSS and 100 μl calcium-3-dye (Molecular Devices) solution, supplemented with 4 mM probenecid. Changes in fluorescence were monitored over a 50 s time course with agonist addition at 17s using a Flexstation (Molecular Devices).

Effect of CRTH2 Agonists on Calcium Mobilisation in CHO-CRTH2 Cells

PGD₂ caused a dose-dependent increase in intracellular Ca²⁺ mobilisation in CHO/CRTH2 cells, with an EC₅₀=2.4+0.5 nM (n=3) (FIG. 1).

Effect of Compounds of General Formula (I) on the Calcium Mobilisation Induced By PGD₂

PGD₂-stimulated Ca²⁺ flux was fully inhibited by the compounds of general formula (I) and the IC₅₀ value for each compound in the calcium assay was comparable to its Ki value in Radioligand binding. IC₅₀ values of compounds of general formula (I) varied from 5 nM to 1 μM. The results for several compounds of general formula (I) are shown in Table 2. Increasing doses of the compounds of general formula (I) caused a dose-dependent and parallel shift of the PGD₂ dose response curve in CHO/CRTH2 cells, thereby indicating that the compounds are competitive CRTH2 antagonists.

The antagonistic effect of the compounds of general formula (I) appears to be CRTH2 selective, since no inhibitory effect was seen with ATP-stimulated Ca²⁺ flux in CHO/CRTH2 cells.

TABLE 2
Inhibition of PGD₂-induced calcium flux

| Compound | CRTH2 Ca flux IC₅₀ (nM) |
|---|---|
| 1 | 280 |
| 2 | 163 |
| 5 | 268 |
| 6 | 345 |
| 7 | 163 |
| 8 | 330 |
| 10 | 79 |
| 11 | 197 |
| 12 | 82 |
| 13 | 1650 |
| 14 | 390 |
| 21 | 1060 |

1. A compound of general formula (I) 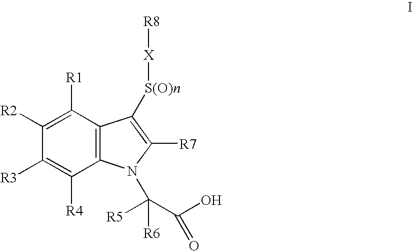
wherein
R¹, R², R³ and R⁴ are independently hydrogen, halo, C₁-C₆ alkyl, —O(C₁-C₆ alkyl), —CON(R⁹)₂, —SOR⁹, —SO₂R⁹, —SO₂N(R⁹)₂, —N(R⁹)₂, —NR⁹COR⁹, —CO₂R⁹, —COR⁹, —SR⁹, —OH, —NO₂ or —CN;
each R⁹ is independently hydrogen or C₁-C₆ alkyl;
R⁵ and R⁶ are each independently hydrogen, or C₁-C₆ alkyl or together with the carbon atom to which they are attached form a C₃-C₇ cycloalkyl group;
R⁷ is hydrogen or C₁-C₆ alkyl
n is 1 or 2;
X is a bond or, when n is 2, X may also be a NR⁹ group;
wherein R⁹ is as defined above;
when X is a bond R⁸ is C₁-C₆ alkyl, C₂-C₆ alkenyl, C₂-C₆ alkynyl, biphenyl or a 9-14 membered bicyclic or tricyclic heteroaryl group;
when X is a NR⁹ group R⁸ may additionally be phenyl, naphthyl or a 5-7 membered heteroaromatic ring; and
the R⁸ group is optionally substituted with one or more substituents selected from halo, C₁-C₆ alkyl, —O(C₁-C₆)alkyl, aryl, —O-aryl, heteroaryl, —O-heteroaryl, —CON(R⁹)₂, —SOR⁹, —SO₂R⁹, SO₂N(R⁹)₂, —N(R⁹)₂, —NR⁹COR⁹, —CO₂R⁹, —COR⁹, —SR⁹, —OH, —NO₂ or —CN;
wherein R⁹ is as defined above;
or a pharmaceutically acceptable salt, hydrate, solvate, complex or prodrug thereof.

2-30. (canceled)

31. A compound as claimed in claim 1 wherein, independently or in any combination:
R¹ is halo or hydrogen;
R² is halo or hydrogen;
R³ is halo or hydrogen;
R⁴ is halo or hydrogen.

32. A compound as claimed in claim 1 wherein R¹, R³ and R⁴ are hydrogen and R² is halo.

33. A compound as claimed in claim 32 wherein R² is fluoro.

34. A compound as claimed in claim 1 wherein R⁵ and R⁶ are each independently hydrogen or C₁-C₄ alkyl.

35. A compound as claimed in claim 34 wherein at least one of R⁵ and R⁶ are hydrogen.

36. A compound as claimed in claim 35 wherein both R⁵ and R⁶ are hydrogen.

37. A compound as claimed in claim 1 wherein R⁷ is H or C₁-C₆ alkyl.

38. A compound as claimed in claim 37 wherein R⁷is methyl.

39. A compound as claimed in claim 1 wherein n is 2.

40. A compound as claimed in claim 1 wherein X is a bond and R⁸ is C₁-C₆ alkyl, biphenyl or a bicyclic heteroaryl group, any of which may be substituted with halogen, phenyl, —CO₂R⁹ CON(R⁹)₂ or —SO₂R⁹, where R⁹ is as defined in claim 1.

41. A compound as claimed in claim 41 wherein R⁸ is selected from the group consisting of a C₁-C₄ alkyl, biphenyl, and a bicyclic heteroaryl group, any of which may be substituted with phenyl, —CO₂R⁹ CON(R⁹)₂ or —SO₂R⁹, where R⁹ is H or C₁-C₄ alkyl.

42. A compound as claimed in claim 1 wherein X is NR⁹, R⁹ is H or methyl and R⁸ is selected from the group consisting of:
phenyl optionally substituted with one or more halo, C₁-C₆ alkyl or —O(C₁-C₆ alkyl) groups;
C₁-C₆ alkyl, optionally substituted with aryl; and
heteroaryl.

43. A compound as claimed in claim 42, wherein R⁸ is selected from the group consisting of phenyl, benzyl or pyridyl, any of which may optionally be substituted with one or more halo, methyl or methoxy groups.

44. A compound of general formula (II): 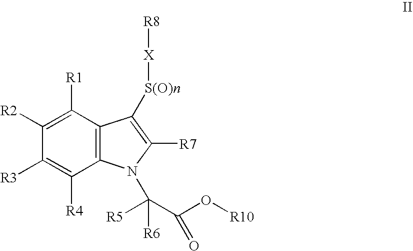
wherein R¹, R², R³, R⁴, R⁵, R⁶, n, X, R⁷ and R⁸ are as defined for general formula (I); R¹⁰ is C₁-C₆ alkyl, aryl, (CH₂)ₘOC(═O)C₁-C₆alkyl, (CH₂)ₘN(R¹¹ )₂, CH((CH₂)ₘO(C═O)R¹²)₂;
m is 1 or 2;
R¹¹ is hydrogen or methyl;
R¹² is C₁-C₁₈ alkyl.

45. A compound as claimed in claim 15 wherein, independently or in any combination:
R¹ is halo or hydrogen;
R² is halo or hydrogen;
R³ is halo or hydrogen;
R⁴ is halo or hydrogen.

46. A compound as claimed in claim 44 wherein R¹, R³ and R⁴ are hydrogen and R² is halo.

47. A compound as claimed in claim 46 wherein R² is fluoro.

48. A compound as claimed in claim 44 wherein R⁵ and R⁶ are each independently hydrogen or C₁-C₄ alkyl.

49. A compound as claimed in claim 48 wherein at least one of R⁵ and R⁶ are hydrogen.

50. A compound as claimed in claim 38 wherein both R⁵ and R⁶ are hydrogen.

51. A compound as claimed in claim 44 wherein R⁷ is H or C₁-C₆ alkyl.

52. A compound as claimed in claim 51 wherein R⁷ is methyl.

53. A compound as claimed in claim 44 wherein n is 2.

54. A compound as claimed in claim 44 wherein X is a bond and R⁸ is C₁-C₆ alkyl, biphenyl or a bicyclic heteroaryl group, any of which may be substituted with halogen, phenyl, —CO₂R⁹ CON(R⁹)₂ or —SO₂R⁹, where R⁹ is as defined in claim 1.

55. A compound as claimed in claim 54 wherein R is selected from the group consisting of a C₁-C₄ alkyl, biphenyl, a bicyclic heteroaryl group and a 5-7 membered heterocyclic ring, any of which may be substituted with phenyl, —CO₂R⁹ CON(R⁹)₂ or —SO₂R⁹, where R⁹ is H or C₁-C₄ alkyl.

56. A compound as claimed in claim 44 wherein X is NR⁹, R⁹ is H or methyl and R⁸ is selected from the group consisting of:
phenyl optionally substituted with one or more halo, C₁-C₆ alkyl or —O(C₁-C₆ alkyl) groups;
C₁-C₆ alkyl, optionally substituted with aryl; and
heteroaryl.

57. A compound as claimed in claim 56, wherein R⁸ is selected from the group consisting of phenyl, benzyl or pyridyl, any of which may optionally be substituted with one or more halo, methyl or methoxy groups.

58. A compound selected from the group consisting of:
[3-(Butane-1-sulfonyl)-5-fluoro-2-methyl-indol-1-yl]-acetic acid
3-(Biphenyl-4-sulfonyl)-5-fluoro-2-methyl-indol-1-yl]-acetic acid
(3-Carboxymethanesulfonyl-5-fluoro-2-methyl-indol-1-yl)-acetic acid
(3-Carbamoylmethanesulfonyl-5-fluoro-2-methyl-indol-1-yl)-acetic acid
[5-Fluoro-3-(2-methanesulfonyl-ethanesulfonyl)-2-methyl-indol-1-yl]-acetic acid
[3-(Benzothiazole-2-sulfonyl)-5-fluoro-2-methyl-indol-1-yl]-acetic acid
[3-(Benzothiazole-2-sulfinyl)-5-fluoro-2-methyl-indol-1-yl]-acetic acid
[5-Fluoro-2-methyl-3-(quinoline-2-sulfonyl)-indol-1-yl]-acetic acid
[5-Fluoro-2-methyl-3-(quinolin-8-ylsulfonyl)-indol-1-yl]-acetic acid
(5-Fluoro-2-methyl-3-phenylmethanesulfonyl-1H-indol-1-yl)-acetic acid
[3-(4-Chloro-phenylsulfamoyl)-5-fluoro-2-methyl-indol-1-yl]-acetic acid
[3-(3-Chloro-phenylsulfamoyl)-5-fluoro-2-methyl-indol-1-yl]-acetic acid
[3-(4-Fluoro-phenylsulfamoyl)-5-fluoro-2-methyl-indol-1-yl]-acetic acid
[3-(2-Chloro-phenylsulfamoyl)-5-fluoro-2-methyl-indol-1-yl]-acetic acid
(3-Benzylsulfamoyl-5-fluoro-2-methyl-indol-1-yl)-acetic acid
[5-Fluoro-3-(2-methoxy-phenylsulfamoyl)-2-methyl-indol-1-yl]-acetic acid
[5-Fluoro-3-(4-methoxy-phenylsulfamoyl)-2-methyl-indol-1-yl]-acetic acid
(5-Fluoro-2-methyl-3-phenylsulfamoyl-indol-1-yl)-acetic acid
[3-(3,4-Dichloro-benzylsulfamoyl)-5-fluoro-2-methyl-indol-1-yl]-acetic acid
[5-Fluoro-3-(3-methoxy-phenylsulfamoyl)-2-methyl-indol-1-yl]-acetic acid
(5-Fluoro-2-methyl-3-m-tolylsulfamoyl-indol-1-yl)-acetic acid
(5-Fluoro-2-methyl-3-p-tolylsulfamoyl-indol-1-yl)-acetic acid
[3-(4-Chloro-benzylsulfamoyl)-5-fluoro-2-methyl-indol-1-yl]-acetic acid
[3-(Benzyl-methyl-sulfamoyl)-5-fluoro-2-methyl-indol-1-yl]-acetic acid
[5-Fluoro-2-methyl-3-(pyridin-3-ylsulfamoyl)-indol-1-yl]-acetic acid;
and the C₁-C₆ alkyl, aryl, (CH₂)ₘOC(═O)C₁-C₆alkyl, (CH₂)ₘN(R¹¹)₂, CH((CH₂)ₘO(C═O)R¹²)₂ esters of any of the above; wherein
m is 1 or 2;
R¹¹ is hydrogen or methyl;
R¹² is C₁-C₁₈ alkyl.

59. A process for the preparation of a compound of general formula (I) as claimed claim 1 and wherein n is 1 or 2 and X is a bond, the process comprising treating a compound of general formula (Ia), which is a compound of general formula (I) wherein n is 0 and X is a bond, by oxidation with a suitable oxidising agent.

60. A process for the preparation of a compound of general formula (I) as claimed in claim 1, the process comprising reacting a compound of general formula (II) as defined in claim 44 and wherein R¹⁰ is C₁-C₆ alkyl with a base.

61. A method for the treatment of a disease or condition mediated by the action of PGD₂ at the CRTH2 receptor, the method comprising administering to a patient in need of such treatment a compound as claimed in claim 1 or a compound as claimed in claim 44.

62. The method of claim 61, further comprising administering to the patient one or more additional active agents useful in the treatment of diseases and conditions mediated by PGD₂ at the CRTH2 receptor.

63. The method of claim 62 wherein the additional active agents are selected from the group consisting of β2 agonists, corticosteroids, antihistamines, leukotriene antagonists, anti-IgE antibody therapies, anti-infectives, anti-fungals, immunosuppressants, other antagonists of PGD₂ acting at other receptors, inhibitors of phoshodiesterase type 4, drugs that modulate cytokine production, drugs that modulate the activity of Th2 cytokines IL-4 and IL-5, PPAR-γ agonists and 5-lipoxygenase.

64. The method of claim 63, wherein the additional active agents are selected from the group consisting of salmeterol, fluticasone, loratidine, montelukast, omalizumab, fusidic acid, clotrimazole, tacrolimus, pimecrolimus, DP antagonists, cilonilast, inhibitors of TNFα converting enzyme (TACE), blocking monoclonal antibodies, soluble receptors, rosiglitazone and zileuton.

65. A method for the treatment of a disease or condition selected from the group consisting of allergic asthma, perennial allergic rhinitis, seasonal allergic rhinitis, atopic dermatitis, contact hypersensitivity (including contact dermatitis), conjunctivitis, especially allergic conjunctivitis, eosinophilic bronchitis, food allergies, eosinophilic gastroenteritis, inflammatory bowel disease, ulcerative colitis and Crohn's disease, mastocytosis, autoimmune diseases such as hyper IgE syndrome and systemic lupus erythematus, psoriasis, acne, multiple sclerosis, allograft rejection, reperfusion injury and chronic obstructive pulmonary disease; or rheumatoid arthritis, psoriatic arthritis and osteoarthritis, the method comprising administering to a patient in need of such treatment a compound as claimed in claim 1 or a compound as claimed in claim 44.

66. The method of claim 65, further comprising administering to the patient one or more additional active agents useful in the treatment of diseases and conditions mediated by PGD₂ at the CRTH2 receptor.

67. The method of claim 66 wherein the additional active agents are selected from the group consisting of β2 agonists, corticosteroids, antihistamines, leukotriene antagonists, anti-IgE antibody therapies, anti-infectives, anti-fungals, immunosuppressants, other antagonists of PGD₂ acting at other receptors, inhibitors of phoshodiesterase type 4, drugs that modulate cytokine production, drugs that modulate the activity of Th2 cytokines IL-4 and IL-5, PPAR-γ agonists and 5-lipoxygenase.

68. The method of claim 67, wherein the additional active agents are selected from the group consisting of salmeterol, fluticasone, loratidine, montelukast, omalizumab, fusidic acid, clotrimazole, tacrolimus, pimecrolimus, DP antagonists, cilonilast, inhibitors of TNFA converting enzyme (TACE), blocking monoclonal antibodies, soluble receptors, rosiglitazone and zileuton.

69. A pharmaceutical composition comprising a compound as claimed in claim 1 together with a pharmaceutical excipient or carrier.

70. A composition as claimed in claim 69 formulated for oral, rectal, nasal, bronchial (inhaled), topical (including eye drops, buccal and sublingual), vaginal or parenteral (including subcutaneous, intramuscular, intravenous and intradermal) administration.

71. A composition as claimed in claim 70 formulated for oral, nasal, bronchial or topical administration.

72. A composition as claimed in claim 69 containing one or more additional active agents useful in the treatment of diseases and conditions mediated by PGD₂ at the CRTH2 receptor.

73. A composition as claimed in claim 72, wherein the additional active agents are selected from the group consisting of β2 agonists, corticosteroids, antihistamines, leukotriene antagonists, anti-IgE antibody therapies, anti-infectives, anti-fungals, immunosuppressants, other antagonists of PGD₂ acting at other receptors, inhibitors of phoshodiesterase type 4, drugs that modulate cytokine production, drugs that modulate the activity of Th2 cytokines IL-4 and IL-5, PPAR-γ agonists and 5-lipoxygenase.

74. A composition as claimed in claim 73, wherein the additional active agents are selected from the group consisting of salmeterol, fluticasone, loratidine, montelukast, omalizumab, fusidic acid, clotrimazole, tacrolimus, pimecrolimus, DP antagonists, cilonilast, inhibitors of TNFα converting enzyme (TACE), blocking monoclonal antibodies, soluble receptors, rosiglitazone and zileuton.

75. A pharmaceutical composition comprising a compound as claimed in claim 44 together with a pharmaceutical excipient or carrier.

76. A composition as claimed in claim 75 formulated for oral, rectal, nasal, bronchial (inhaled), topical (including eye drops, buccal and sublingual), vaginal or parenteral (including subcutaneous, intramuscular, intravenous and intradermal) administration.

77. A composition as claimed in claim 76 formulated for oral, nasal, bronchial or topical administration.

78. A composition as claimed in claim 46 containing one or more additional active agents useful in the treatment of diseases and conditions mediated by PGD₂ at the CRTH2 receptor.

79. A composition as claimed in claim 78, wherein the additional active agents are selected from the group consisting of β2 agonists, corticosteroids, antihistamines, leukotriene antagonists, anti-IgE antibody therapies, anti-infectives, anti-fungals, immunosuppressants, other antagonists of PGD₂ acting at other receptors, inhibitors of phoshodiesterase type 4, drugs that modulate cytokine production, drugs that modulate the activity of Th2 cytokines IL-4 and IL-5, PPAR-γ agonists and 5-lipoxygenase.

80. A composition as claimed in claim 79, wherein the additional active agents are selected from the group consisting of salmeterol, fluticasone, loratidine, montelukast, omalizumab, fusidic acid, clotrimazole, tacrolimus, pimecrolimus, DP antagonists, cilonilast, inhibitors of TNFα converting enzyme (TACE), blocking monoclonal antibodies, soluble receptors, rosiglitazone and zileuton.
